(19) United States
(12) Patent Application Publication
(10) Pub. No.: US 2024/0319178 A1
(43) Pub. Date: Sep. 26, 2024

(54) COMPOUNDS, KITS, AND METHODS FOR BEAD-BASED DETECTION OF ANTI-MICROBE ANTIBODIES

(71) Applicant: Quest Diagnostics Investments LLC, Secaucus, NJ (US)

(72) Inventor: Mary Lapé Nixon, Anaheim, CA (US)

(73) Assignee: Quest Diagnostics Investments LLC, Secaucus, NJ (US)

(21) Appl. No.: 18/270,429

(22) PCT Filed: Dec. 29, 2021
(86) PCT No.: PCT/US2021/065565

§ 371 Date: Jun. 29, 2023

Related U.S. Application Data
(60) Provisional application No. 63/132,242, filed on Dec. 30, 2020.

(51) Int. Cl. G01N 33/543; G01N 33/545; G01N 33/569; G01N 33/576

(52) U.S. Cl. CPC G01N 33/54313; G01N 33/545; G01N 33/56911; G01N 33/5765; G01N 2333/08; G01N 2333/20; G01N 2469/20

(57) ABSTRACT

The present disclosure relates generally an immunoassay platform that utilizes a bead-based system to detect the presence or absence of antibodies in a test sample. The disclosed platform relates to methods and kits and can be used to detect the presence, absence, or exposure to Borrelia miyamotoi or Hepatitis D virus (HDV), or other pathogens or microbes of interest in a subject.

### CROSS-REFERENCE TO RELATED APPLICATIONS

This application claims the benefit of and priority to U.S. Provisional Application No. 63/132,242, filed Dec. 30, 2020, the disclosure of which is incorporated by reference herein in its entirety.

### FIELD OF INVENTION

The present disclosure relates generally an immunoassay platform that utilizes a bead-based system to detect the presence or absence of antibodies in a test sample. The disclosed platform relates to methods and kits and can be used to detect the presence, absence, or exposure to Borrelia miyamotoi or Hepatitis D virus (HDV), or other pathogens or microbes of interest in a subject.

### BACKGROUND

The following discussion is merely provided to aid the reader in understanding the disclosure and is not admitted to describe or constitute prior art thereto.

Borrelia miyamotoi is a type of spiral-shaped bacteria that is closely related to the bacteria that cause tick-borne relapsing fever (TBRF). It is more distantly related to the bacteria that cause Lyme disease. First identified in 1995 in ticks from Japan, B. miyamotoi has since been detected in two types of North American ticks, the blacklegged or “deer” tick (Ixodes scapularis) and the Western blacklegged tick (Ixodes pacificus). These ticks are already known to spread the germs that cause several diseases, including Lyme disease and anaplasmosis. B. miyamotoi infections are generally treated with a 2- to 4-week course of the antibiotic doxycycline, although amoxicillin and ceftriaxone have also been successfully used.

Patients with a B. miyamotoi infection most commonly present with symptoms such as fever, chills, headache, body and joint pain, and fatigue. Unlike Lyme disease, development of a rash is uncommon and occurs in fewer than 1 in 10 patients.

Currently, diagnostic testing for the presence of a B. miyamotoi infection is limited to polymerase chain reaction (PCR)-based detection of DNA from the bacteria or enzyme-linked immunosorbent assay (ELISA)-based detection of antibodies to the bacteria. Both existing methods suffer from severe limitations, including a high false positive rate and a lack of sensitivity.

Hepatitis D virus (HDV) causes an infection of the liver. Hepatitis D only occurs in people who are also infected with the hepatitis B virus. Hepatitis D is spread when blood or other body fluids from a person infected with the virus enters the body of someone who is not infected. Hepatitis D can be an acute, short-term infection or become a long-term, chronic infection. Hepatitis D can cause severe symptoms and serious illness that can lead to life-long liver damage and even death. People can become infected with both hepatitis B and hepatitis D viruses at the same time (known as “coinfection”) or get hepatitis D after first being infected with the hepatitis B virus (known as “superinfection”). There is no vaccine to prevent hepatitis D.

Because cases of hepatitis D are not clinically distinguishable from other types of acute viral hepatitis, diagnosis can be confirmed only by testing for the presence of antibodies against HDV and/or HDV RNA. Both existing methods suffer from severe limitations, including a high false positive rate and a lack of sensitivity.

Accordingly, there is a need in the art for methods of detecting B. miyamotoi and HDV infection or exposure, as well as other bacteria and viruses where current approaches are less accurate and/or sensitive. The present disclosure fulfills that need by providing a bead-based system to detect the presence or absence of anti-microbe antibodies in a test sample as a way of establishing infection or exposure to B. miyamotoi and HDV or other pathogens or microbes of interest.

### SUMMARY

Described herein are novel methods, compositions, and kits for detecting the presence, absence, or exposure to pathogens or microbes, such as B. miyamotoi and HDV.

In a first aspect, the present disclosure provides a method of determining exposure of a subject to a pathogen or microbe, comprising (i) incubating a liquid biological sample obtained from a subject with a bead particle comprising a core bead coupled to a plurality of antibodies bound to an antigen derived from a pathogen or microbe of interest, such that the antigen is presented on the outside of the bead particle and any antibodies in the liquid biological sample that recognize the antigen can bind to the bead particle; (ii) washing the bead particle; (iii) incubating the bead particle with a detectably labeled antibody that binds to an antibody of the species of the subject; and determining the presences or absence of the detectably labeled antibody, wherein the presence of the detectable label indicates the subject was exposed to the pathogen or microbe of interest and the absence of the detectable label indicates the subject was not exposed to the pathogen or microbe of interest.

In some embodiments of the first aspect, the subject is a human.

In some embodiments of the first aspect, the detectably labeled antibody specifically binds to IgG and/or IgM. In some embodiments of the first aspect, the detectable label is selected from among a fluorophore, a pigment, a radioactive isotope, a chemiluminescent molecule, a chromophore, an electron dense label, an enzyme, a dye, a metal, biotin, avidin, streptavidin, and a hapten.

In some embodiments of the first aspect, the pathogen or microbe of interest is a virus, bacteria, or fungi.

In some embodiments of the first aspect, the antibodies coupled to the core bead are anti-His antibodies and the antigen is His-tagged. As explained in more detail herein, a His-tag is only one exemplary tag, and it should be understood that other tag sequences can also be used to bind the antigen of interest to the bead particle. In some embodiments of the first aspect, the antigen was produced recombinately from a bacterial host.

In some embodiments of the first aspect, the core bead is a polystyrene microparticle.

In some embodiments of the first aspect, the liquid biological sample is selected from among blood, serum, and plasma. In some embodiments of the first aspect, the liquid biological sample is diluted serum.

In some embodiments of the first aspect, the amount of the detectable label determined to be present in the liquid biological sample correlates to the amount of anti-pathogen or anti-microbe IgG and/or IgM in the liquid biological sample.

In a second aspect, the present disclosure provides a bead particle comprising a microparticle coupled to a plurality of antibodies bound to an antigen derived from a pathogen or microbe of interest; wherein the antigen comprises a tag sequence and wherein the antibodies specifically bind to the tag sequence.

In some embodiments of the second aspect, the microparticle is a polystyrene microparticle.

In some embodiments of the second aspect, the pathogen or microbe of interest is a virus, bacteria, or fungi.

In some embodiments of the second aspect, the tag sequence is a His-tag. In some embodiments of the second aspect, the antigen was produced recombinantly from a bacterial host.

In a third aspect, the disclosure provides methods of determining exposure of a subject to Borrelia miyamotoi, comprising (i) incubating a liquid biological sample obtained from a subject with a bead particle comprising a core bead coupled to a plurality of antibodies bound to a B. miyamotoi glycerophosphodiester phosphodiesterase (glpQ), such that any anti-glpQ antibodies in the liquid biological sample can bind to the glpQ on the bead particle; (ii) washing the bead particle; (iii) incubating the bead particle with a detectably labeled antibody that binds to an antibody of the species of the subject; and determining the presences or absence of the detectably labeled antibody, wherein the presence of the detectable label indicates the subject was exposed to B. miyamotoi and the absence of the detectable label indicates the subject was not exposed to B. miyamotoi.

In some embodiments of the third aspect, the subject is a human.

In some embodiments of the third aspect, the detectably labeled antibody specifically binds to IgG and/or IgM. In some embodiments of the third aspect, the detectable label is selected from among a fluorophore, a pigment, a radioactive isotope, a chemiluminescent molecule, a chromophore, an electron dense label, an enzyme, a dye, a metal, biotin, avidin, streptavidin, and a hapten.

In some embodiments of the third aspect, the antibodies bound to the B. miyamotoi glpQ specifically bind to B. miyamotoi glpQ. In some embodiments of the third aspect, the antibodies are bound to a tag sequence or tag domain on the B. miyamotoi glpQ. In some embodiments of the third aspect, the antibodies bound to the B. miyamotoi glpQ are anti-histidine antibodies and the B. miyamotoi glpQ is His-tagged. In some embodiments of the third aspect, the B. miyamotoi glpQ was produced recombinately from a bacterial host.

In some embodiments of the third aspect, the core bead is a polystyrene microparticle.

In some embodiments of the third aspect, the liquid biological sample is selected from among blood, serum, and plasma. In some embodiments of the third aspect, the liquid biological sample is diluted serum.

In some embodiments of the third aspect, the amount of the detectable label determined to be present in the liquid biological sample correlates to the amount of anti-B. miyamotoi IgG and/or IgM in the liquid biological sample.

In a fourth aspect, the disclosure provides a bead particle comprising a microparticle coupled to a plurality of antibodies bound to a B. miyamotoi glycerophosphodiester phosphodiesterase (glpQ).

In some embodiments of the fourth aspect, the microparticles is a polystyrene microparticle.

In some embodiments of the fourth aspect, the antibodies bound to the B. miyamotoi glpQ specifically bind to B. miyamotoi glpQ. In some embodiments of the fourth aspect, the antibodies are bound to a tag sequence or tag domain on the B. miyamotoi glpQ. In some embodiments of the fourth aspect, the antibodies bound to the B. miyamotoi glpQ are anti-histidine antibodies and the B. miyamotoi glpQ is His-tagged. In some embodiments of the fourth aspect, the B. miyamotoi glpQ was produced recombinately from a bacterial host.

In a fifth aspect, the disclosure provides methods of determining exposure of a subject to hepatitis D virus (HDV), comprising (i) incubating a liquid biological sample obtained from a subject with a bead particle comprising a core bead coupled to a plurality of antibodies bound to a HDV antigen, such that any antibodies in the liquid biological sample that are capable of binding the HDV antigen can bind to the HDV antigen on the bead particle; (ii) washing the bead particle; (iii) incubating the bead particle with a detectably labeled antibody that binds to an antibody of the species of the subject; and determining the presences or absence of the detectably labeled antibody, wherein the presence of the detectable label indicates the subject was exposed to HDV and the absence of the detectable label indicates the subject was not exposed to HDV.

In some embodiments of the fifth aspect, the subject is a human.

In some embodiments of the fifth aspect, the detectably labeled antibody specifically binds to IgG and/or IgM. In some embodiments of the fifth aspect, the detectable label is selected from among a fluorophore, a pigment, a radioactive isotope, a chemiluminescent molecule, a chromophore, an electron dense label, an enzyme, a dye, a metal, biotin, avidin, streptavidin, and a hapten.

In some embodiments of the fifth aspect, the antibodies bound to the HDV antigen specifically bind to the HDV antigen. In some embodiments of the fifth aspect, the antibodies are bound to a tag sequence or tag domain on the HDV antigen. In some embodiments of the fifth aspect, the antibodies bound to the HDV antigen are anti-histidine antibodies and the HDV antigen is His-tagged. In some embodiments of the fifth aspect, the HDV antigen was produced recombinately from a bacterial host.

In some embodiments of the fifth aspect, the core bead is a polystyrene microparticle.

In some embodiments of the fifth aspect, the liquid biological sample is selected from among blood, serum, and plasma. In some embodiments of the fifth aspect, the liquid biological sample is diluted serum.

In some embodiments of the fifth aspect, the amount of the detectable label determined to be present in the liquid biological sample correlates to the amount of anti-HDV IgG and/or IgM in the liquid biological sample.

In a sixth aspect, the disclosure provides a bead particle comprising a microparticle coupled to a plurality of antibodies bound to a hepatitis D virus (HDV) antigen.

In some embodiments of the sixth aspect, the microparticles is a polystyrene microparticle.

In some embodiments of the sixth aspect, the antibodies bound to the HDV antigen specifically bind to the HDV antigen. In some embodiments of the sixth aspect, the antibodies are bound to a tag sequence or tag domain on the HDV antigen. In some embodiments of the sixth aspect, the antibodies bound to the HDV antigen are anti-histidine antibodies and the HDV antigen is His-tagged. In some embodiments of the sixth aspect, the HDV antigen was produced recombinately from a bacterial host.

In a seventh aspect, the disclosure provides a kit comprising a bead particle according to any of the foregoing aspects or embodiments.

In some embodiments, the kit may further comprise a detectably labeled antibody that specifically binds to IgG and/or IgM. In some embodiments, the detectably labeled antibody bind to human IgG and/or human IgM. In some embodiments, the detectable label can be selected from among a fluorophore, a pigment, a radioactive isotope, a chemiluminescent molecule, a chromophore, an electron dense label, an enzyme, a dye, a metal, biotin, avidin, streptavidin, and a hapten.

The foregoing general description and following detailed description are exemplary and explanatory and are intended to provide further explanation of the disclosure as claimed. Other objects, advantages, and novel features will be readily apparent to those skilled in the art from the following brief description of the drawings and detailed description of the disclosure.

### DETAILED DESCRIPTION

The present disclosure provides a bead-based system to detect the presence or absence or exposure to a pathogen (e.g., virus, bacteria or fungi) or microbe of interest, as well as methods and kits utilizing the same. The disclosed systems, methods, compositions and kits allow for sensitive detection of a variety of pathogen/microbe exposure by utilizing tag sequences appended to an antigen from the pathogen or microbe of interest.

For example, the present disclosure provides a bead-based system to detect a subject's exposure to B. miyamotoi by determining the presence or absence of anti-B. miyamotoi antibodies in a test sample. Numerous attempts have been made since 2013 to develop immunoassays to detect antibodies to B. miyamotoi using the standard enzyme-linked immunosorbent assay (ELISA). The protein expressed by B. miyamotoi and hence used for antibody detection is the organism's glycerophosphodiester phosphodiesterase (glpQ). However, exhaustive and repeated attempts to develop a sensitive and specific ELISA-based assay invariably resulted in failure due to unacceptable assay performance, most notably poor assay specificity (as evidenced by nonspecific reactivity to other organisms).

The presently disclosed compositions and methods differ from previous attempts by relying on a bead-based (e.g., LUMINEX®) multiplex platform. Moreover, the present methods do not rely on the standard indirect sandwich approach generally used for antibody detection because the inventors determined that B. miyamotoi glpQ is an atypical protein that does not respond well to this method.

The novel methods disclosed herein utilizes glpQ that is tagged with histidine residues in a “His-tag,” which generally comprises 5-10 histidines in a series. In some embodiments, the glpQ may be recombinant and produced in a bacteria (e.g., E. coli) such that the histidine residues are encoded as a by-product of production in a bacterial host. Anti-histidine monoclonal antibodies are bound to beads, such as LUMINEX® beads, via a chemical linkage (e.g., carbodiimide chemistry). These antibody-decorated beads can be used to capture the histidine-tagged glpQ with the anti-histidine antibodies, which efficiently present the antigen to antibodies contained in test serum.

Similar studies have been completed for determining exposure to hepatitis D virus (HDV) using a His-tagged HDV antigen in the place of the His-tagged glpQ. Of course, any tag sequence that is readily recognizable by an antibody could be used as a target epitope for binding an antigen of interest from a pathogen or microbe of interest to a bead particle. Indeed, numerous tag domains and sequences that could feasibly be utilized in the disclosed system are known in the art (e.g., chitin binding protein, maltose binding protein, Strep-tag, glutathione-S-transferase (GST), thioredoxin, poly(NANP), FLAG-tag, ALFA-tag, V5-tag, Myc-tag, HA-tag, Spot-tag, T7-tag and NE-tag, ALFA-tag, AviTag, C-tag, calmodulin-tag, polyglutamate tag, polyarginine tag, E-tag, HA-tag, Myc-tag, NE-tag, S-tag, T7-tag, Ty-tag, V5-tag, and Xpress tag). As a result of using tag domains/sequences instead of antigen-specific antibodies that are bound to a core bead, the present system provide unparalleled versatility and flexibility in selecting an antigen or pathogen of interest.

Studies using this approach showed exquisite assay specificity, as well as excellent sensitivity and reproducibility. The disclosed antibody assays, which may detect IgG and/or IgM, have been fully validated for determining a subject's exposure to bacteria (e.g., B. miyamotoi) and viruses (e.g., hepatitis D virus), and therefore it should be understood that the disclosed platform can be used to detect or determine exposure to any pathogen or microbe of interest that may have elicited an immune response in a subject.

### I. Definitions

It is to be understood that methods are not limited to the particular embodiments described, and as such may vary. It is also to be understood that the terminology used herein is for the purpose of describing particular embodiments only, and is not intended to be limiting. The scope of the present technology will be limited only by the appended claims.

For the purpose of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B, and C).

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, “about” means plus or minus 10% as well as the specified number. For example, “about 10” should be understood as both “10” and “9-11.”

As used herein, “optional” or “optionally” means that the subsequently described event or circumstance may or may not occur, and that the description includes instances where said event or circumstance occurs and instances where it does not.

As used herein, “a plurality of antibodies” means at least one antibody. In other words, “a plurality of antibodies” should be understood as meaning 1, 2, 3, 4, 5, 6, 7, 8, 9, or 10 or more antibodies. “A plurality of antibodies” may be 1-10, 2-10, 3-10, 4-10, 5-10, 1-9, 2-9, 3-9, 4-9, 5-9, 1-8, 2-8, 3-8, 4-8, 5-8, 1-7, 2-7, 3-7, 4-7, 5-7, 1-6, 2-6, 3-6, 4-6, or 5-6 antibodies.

### II. Antibody-Decorated Beads

Provided herein are antibody-decorated beads (i.e., “bead particles”), which comprise a core bead coupled to at least one or a plurality of antibodies bound to an antigen from a pathogen or microbe of interest, such as, B. miyamotoi glycerophosphodiester phosphodiesterase (glpQ) or a hepatitis D virus (HDV) antigen. For the purposes of the present disclosure, references to a bead, bead particle, or microparticle as “decorated” indicated that a plurality of antibodies (i.e., 1, 2, 3, 4, 5, 6, 7, 8, 9, or 10 or more) are bound to the outside or surface of the bead, bead particle, or microparticle. The way in which the antibodies are bound is not particularly limited and may include, for example, a chemical linkage (e.g., carbodiimide chemistry).

The disclosed bead particles can be used in assays to detect whether antibodies specific for B. miyamotoi, HDV, or any other pathogen (e.g., virus, bacteria, or fungi) or microbe of interest are present in a liquid sample (e.g., a biological sample such as blood, plasma, saliva, etc.). In order for the bead particles to function for detection, the antibodies decorated on the outside of the bead and the antigen (e.g., glpQ or a HDV antigen) bound to the antibody must be arranged such that any antibodies in the liquid sample can bind to the antigen (e.g., anti-glpQ antibodies or anti-HDV antibodies) on the bead particle. After being contacted with a sample of interest, the bead particle can be washed and subsequently incubated with a detectably labeled antibody that binds to an antibody of the species from which the sample was derived (i.e., the “subject”). Binding of the detectably labeled antibody to the bead particle indicates that the subject was or is exposed to the microbe or pathogen of interest, such as B. miyamotoi, HDV, or any other virus, bacteria, or fungi of interest that has an antigen presented on the bead.

The antibodies decorated on the outside of a core bead particle must be capable of binding to an antigen from the pathogen of interest (e.g., a B. miyamotoi glpQ, a HDV antigen, etc.), and it some instances the antibodies decorated on the outside of a core bead particle may specifically bind to that antigen. However, the antigen from the pathogen of interest (e.g., a B. miyamotoi glpQ, a HDV antigen, etc.) may also comprise a peptide tag (i.e., a tag sequence or tag domain), such as a Histidine tag (i.e., “His-tag”) to which the antibodies decorated on the outside of a core bead can bind. Other known tag sequences or tag domains that may be used include, but are not limited to, chitin binding protein (CBP), maltose binding protein (MBP), Strep-tag (WSHPQFEK), glutathione-S-transferase (GST), thioredoxin (TRX), poly(NANP), FLAG-tag (DYKDDDDK), ALFA-tag, V5-tag, Myc-tag, HA-tag, Spot-tag, T7-tag and NE-tag, ALFA-tag, AviTag, C-tag, calmodulin-tag, polyglutamate tag (e.g., EEEEEE or 5-10 Es), polyarginine tag (e.g., 5-10 Rs), E-tag (GAPVPYPDPLEPR), HA-tag (YPYDVPDYA), Myc-tag (EQKLISEEDL), NE-tag (TKENPRSNQEESYDDNES), S-tag (KETAAAKFERQHMDS), T7-tag (MASMTGGQQMG), Ty-tag (EVHTNQDPLD), V5-tag (GKPIPNPLLGLDST), and Xpress tag (DLYDDDDK). Utilizing a peptide tag like a His-tag provides benefits in scaling and a capability to utilize a target other than B. miyamotoi glpQ or a HDV antigen, as a His-tag (or other tag sequence) can readily be appended to B. miyamotoi glpQ, the HDV antigen, or any other peptide/antigen target during recombinant expression of the antigen in a recombinant host, such as a bacterial. In some embodiments, the antigen from the pathogen of interest (e.g., a B. miyamotoi glpQ, a HDV antigen, etc.) bound to the antibodies decorated on the outside of the core bead is a recombinant antigen and in some embodiments, the recombinant antigen comprises a His-tag or another tag sequence. Accordingly, in some embodiments, the antibody decorated on the outside of the core particle may be an anti-histidine (i.e., anti-His-tag) antibody. In some embodiments, the antibody decorated on the outside of the core particle may be an antibody that binds to chitin binding protein, maltose binding protein, Strep-tag, glutathione-S-transferase (GST), thioredoxin, poly(NANP), FLAG-tag, ALFA-tag, V5-tag, Myc-tag, HA-tag, Spot-tag, T7-tag and NE-tag, ALFA-tag, AviTag, C-tag, calmodulin-tag, polyglutamate tag, polyarginine tag, E-tag, HA-tag, Myc-tag, NE-tag, S-tag, T7-tag, Ty-tag, V5-tag, or Xpress tag or any other known tag sequence.

The core bead can be any bead suitable for bead-based assays. For instance, polystyrene beads are a commonly used solid support for bead-based assays. In some embodiments, the core bead of the bead particle may be a polystyrene microparticle, such as a LUMINEX® bead. Although generally spherical, the shape of the core bead is not particularly limited, and may be cylindrical, cubic, or another shape aside from spherical. In some embodiments, the core bead is spherical. The size of the core bead vary also vary, and in some embodiments the diameter may be between 0.1 μm and 5 mm. For example, in some embodiments, the core bead may have a diameter of about 0.1 μm, about 0.2 μm, about 0.3 μm, about 0.4 μm, about 0.5 μm, about 0.6 μm, about 0.7 μm, about 0.8 μm, about 0.9 μm, about 1.0 μm, about 1.5 μm, about 2.0 μm, about 2.5 μm, about 3.0 μm, about 3.5 μm, about 4.0 μm, about 4.5 μm, about 5.0 μm, about 5.5 μm, about 6.0 μm, about 6.5 μm, about 7.0 μm, about 7.5 μm, about 8.0 μm, about 8.5 μm, about 9.0 μm, about 9.5 μm, about 10.0 μm, about 10.5 μm, about 11.0 μm, about 11.5 μm, about 12.0 μm, about 12.5 μm, about 13.0 μm, about 13.5 μm, about 14.0 μm, about 14.5 μm, about 15.0 μm, about 15.5 μm, about 16.0 μm, about 16.5 μm, about 17.0 μm, about 17.5 μm, about 18.0 μm, about 18.5 μm, about 19.0 μm, about 19.5 μm, about 20.0 μm, about 20.5 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, about 125 μm, about 150 μm, about 175 μm, about 200 μm, about 225 μm, about 250 μm, about 275 μm, about 300 μm, about 325 μm, about 350 μm, about 375 μm, about 400 μm, about 425 μm, about 450 μm, about 475 μm, about 500 μm, about 550 μm, about 600 μm, about 650 μm, about 700 μm, about 750 μm, about 800 μm, about 850 μm, about 900 μm, about 950 μm, about 1 mm, about 1.25 mm, about 1.5 mm, about 1.75 mm, about 2 mm, about 2.25 mm, about 2.5 mm, about 2.75 mm, about 3 mm, about 3.25 mm, about 3.5 mm, about 3.75 mm, about 4 mm, about 4.25 mm, about 4.5 mm, about 4.75 mm, or about 5 mm.

In some embodiments, the disclosed bead particle comprises a microparticle coupled to a plurality of antibodies bound to a B. miyamotoi glycerophosphodiester phosphodiesterase (glpQ). In some embodiments, the microparticle is a polystyrene microparticle. In some embodiments, the antibodies bound to the B. miyamotoi glpQ specifically bind to B. miyamotoi glpQ, while in some embodiments, the antibodies bound to the B. miyamotoi glpQ are anti-histidine antibodies and the B. miyamotoi glpQ is His-tagged. In some embodiments, the B. miyamotoi glpQ was produced recombinately from a bacterial host (e.g., E. coli).

In some embodiments, the disclosed bead particle comprises a microparticle coupled to a plurality of antibodies bound to a HDV antigen. In some embodiments, the microparticle is a polystyrene microparticle. In some embodiments, the antibodies bound to the HDV antigen specifically bind to the HDV antigen, while in some embodiments, the antibodies bound to the HDV antigen are anti-histidine antibodies and the HDV antigen is His-tagged. In some embodiments, the HDV antigen was produced recombinantly from a bacterial host (e.g., E. coli).

In some embodiments, the disclosed bead particle comprises a microparticle coupled to a plurality of anti-histidine antibodies that are bound to a His-tagged antigen of interest. In some embodiments, the disclosed bead particle comprises a microparticle coupled to a plurality of antibodies that bind to a specific tag sequence and which are bound to an antigen comprising the same specific tag. When such beads are incubated with a biological sample of interest obtained from a subject (e.g., a human patient), if the biological sample contains antibodies that bind to the antigen of interest, then the presence of those antigen-specific antibodies can be determined by washing the beads and subsequently incubating the beads with detectably-labeled antibodies that are species-specific to the subject (e.g., anti-human IgG or anti-human IgM).

### III. Methods of Detecting Exposure to a Pathogen or Microbe

The disclosed methods can be used to detect the presence, absence, or exposure to any pathogen (e.g., bacteria, virus, or fungi) or microbe of interest with respect to a given subject, so long as the pathogen or microbe of interest elicits an immune response in the subject. By detecting the presence or absence in a sample of IgG and/or IgM that bind to an antigen from a pathogen or microbe of interest, the disclosed methods can determine whether the subject has previously been exposed to that pathogen or microbe.

a. Methods of Detecting Exposure to B. miyamotoi

Numerous attempts have been made to develop immunoassays to detect antibodies to Borrelia miyamotoi using the standard enzyme-linked immunosorbent assay (ELISA), but to date these assays have largely failed or performed inadequately for clinical practice. B. miyamotoi expresses a protein known as glycerophosphodiester phosphodiesterase (glpQ), which has been used as an antibody target for prior ELISA-based detection methods. These prior methods almost invariably suffered from poor assay specificity, which was evidenced by nonspecific reactivity to other organisms.

In contrast, the presently disclosed methods utilize a bead-based multiplex platform; which proved superior to the standard and conventional ELISA-based approach to detection. The disclosed method does not rely on a standard indirect sandwich approach, which is generally used for antibody detection, because the present inventors realized that glpQ is an atypical protein/target that is not conducive for such an approach. Instead, the disclosed methods present glpQ on the outside of a bead particle by binding glpQ to antibodies coupled or attached (i.e., “decorated”) to the outside of a core bead, such as a polystyrene bead or microparticle.

For ease of use, the glpQ may be recombinant and it may be tagged with Histidine residues (i.e., comprise a “His-tag”), for example, as a by-product of its production in a bacterial host, such as Escherichia coli (E. coli). Monoclonal antibodies against histidine residues or His-tags are widely commercially available, and they are commonly used by commercial manufacturers to detect the efficacy of recombinant protein recovery. Moreover, molecular tags, like His-tags, provide the disclosed platform with a versatility of antigen presentation on the disclosed bead particles, as the disclosed methods can be readily adapted to another pathogen or microbe of interest by recombinately expressing an antigen from the pathogen or microbe with a molecular tag like a His-tag.

As noted above, the one or more antibodies (i.e., plurality of antibodies) coupled to the outside of the core bead (e.g., a LUMINEX® bead or polystyrene bead) can be attached to the core bead via any suitable means, such as by carbodiimide chemistry. If anti-His antibodies are decorated on the core bead, then capture of the histidine-tagged glpQ (or other antigen of interest) by these antibodies provides reliable and efficient presentation of the antigen to antibodies contained in test sample, which may be blood, plasma, serum, saliva, etc. Studies using this approach showed exquisite assay specificity, as well as excellent sensitivity and reproducibility.

In general, the disclosed methods of determining exposure of a subject to Borrelia miyamotoi (or another pathogen or microbe of interest), comprises (i) incubating a liquid biological sample obtained from a subject with a bead particle comprising a core bead coupled to a plurality of antibodies bound to a B. miyamotoi glycerophosphodiester phosphodiesterase (glpQ), such that any anti-glpQ antibodies in the liquid biological sample can bind to the glpQ on the bead particle; (ii) washing the bead particle; (iii) incubating the bead particle with a detectably labeled antibody that binds to an antibody of the species of the subject; and determining the presences or absence of the detectably labeled antibody, wherein the presence of the detectable label indicates the subject was exposed to B. miyamotoi and the absence of the detectable label indicates the subject was not exposed to B. miyamotoi.

The subject is a human and another mammal for which it is desired to determine exposure to Borrelia miyamotoi or another pathogen or microbe of interest. The subject will provide a biological sample, which is generally expected to be a liquid. Suitable sample types include, but are not limited to, blood, plasma, serum, or saliva. In some embodiments, the biological sample may be diluted serum.

The detectably labeled antibody (i.e., secondary antibody) that is incubated with the bead particles to indicate whether the subject possesses antibodies that bind to glpQ. The source of the detectably labeled antibody or secondary antibody is not limited, and may be, for example, human or humanized or may be derived from mouse, rat, sheep, horse, pig, cow, camelid, or other mammal. In some embodiments, detectably labeled antibody or secondary antibody specifically binds to IgG and/or IgM. The detectably labeled antibody or secondary antibody also may bind other types of antibodies as well, so long as the detectably labeled antibody or secondary antibody specifically binds to antibodies from the subject on which the method is being performed. Thus, in some embodiments, the detectably labeled antibody or secondary antibody is an anti-human antibody when the subject on which the method is being performed is a human.

In some embodiments, the detectable label may be selected from among a fluorophore, a pigment (e.g., phycoerythrin), a radioactive isotope, a chemiluminescent molecule, a chromophore, an electron dense label, an enzyme, a dye, a metal, biotin, avidin, streptavidin, and a hapten. However, it should be noted that any detectable label may suffice for the purposes of the disclosed methods.

With respect to the antibody or antibodies that are coupled to the outside of the core bead (i.e., decorated on the core bead), these antibodies may bind directly or indirectly to B. miyamotoi glpQ or any other antigen of interest. For example, in some embodiments, the antibody may specifically bind to B. miyamotoi glpQ, while in some embodiments, the antibody may be an anti-histidine antibody (or another tag-specific antibody) and the B. miyamotoi glpQ (or other antigen) may be His-tagged (or comprise another known tag sequence). When a His-tag is desired on the B. miyamotoi glpQ, one can be easily added by producing the B. miyamotoi glpQ recombinately in a bacterial host like E. coli and ensuring that the recombinant sequence encoding the antigen is followed by a sequence encoding histidine repeats (or another tag sequence). Other molecular tags aside from a His-tag (which generally comprises 5-10 histidines) may be used in the present methods as well. For example, other known molecular tag or affinity tags including, but not limited to, chitin binding protein (CBP), maltose binding protein (MBP), Strep-tag (WSHPQFEK), glutathione-S-transferase (GST), thioredoxin (TRX), poly(NANP), FLAG-tag (DYKDDDDK), ALFA-tag, V5-tag, Myc-tag, HA-tag, Spot-tag, T7-tag and NE-tag, ALFA-tag, AviTag, C-tag, calmodulin-tag, polyglutamate tag (e.g., EEEEEE or 5-10 Es), polyarginine tag (e.g., 5-10 Rs), E-tag (GAPVPYPDPLEPR), HA-tag (YPYDVPDYA), Myc-tag (EQKLISEEDL), NE-tag (TKENPRSNQEESYDDNES), S-tag (KETAAAKFERQHMDS), T7-tag (MASMTGGQQMG), Ty-tag (EVHTNQDPLD), V5-tag (GKPIPNPLLGLDST), and Xpress tag (DLYDDDDK). The foregoing and any other peptide tag sequence known in the art may also be suitable for the disclosed methods.

The core bead for the bead particle can also be any bead suitable for multiplex assay platforms like the one described. In particular, polystyrene beads or microparticles are commonly used in this type of assay, and in some embodiments, the core bead may be a polystyrene bead or microparticle. In some embodiments, the polystyrene bead or microparticle may be a LUMINEX® bead or microparticle.

In some embodiments, the amount of the detectable label determined to be present in the liquid biological sample correlates to the amount of anti-B. miyamotoi IgG and/or IgM in the liquid biological sample.

b. Methods of Detecting Exposure to Hepatitis D Virus (HDV)

The presently disclosed a bead-based multiplex platform provides superior detection than a conventional ELISA-based approach for detecting exposure to HDV as well. The disclosed method does not rely on a standard indirect sandwich approach, which is generally used for antibody detection, and instead the disclosed methods present a HDV antigen on the outside of a bead particle by binding the HDV antigen to antibodies coupled or attached (i.e., “decorated”) to the outside of a core bead, such as a polystyrene bead or microparticle.

For ease of use, the HDV antigen may be recombinant and it may be tagged with Histidine residues (i.e., comprise a “His-tag”), for example, as a by-product of its production in a bacterial host, such as Escherichia coli (E. coli). Monoclonal antibodies against histidine residues or His-tags are widely commercially available, and they are commonly used by commercial manufacturers to detect the efficacy of recombinant protein recovery. Moreover, molecular tags, like His-tags, provide the disclosed platform with a versatility of antigen presentation on the disclosed bead particles, as the disclosed methods can be readily adapted to another pathogen or microbe of interest by recombinately expressing an antigen from the pathogen or microbe with a molecular tag like a His-tag.

As noted above, the one or more antibodies (i.e., plurality of antibodies) coupled to the outside of the core bead (e.g., a LUMINEX® bead or polystyrene bead) can be attached to the core bead via any suitable means, such as by carbodiimide chemistry. If anti-His antibodies are decorated on the core bead, then capture of the histidine-tagged HDV antigen (or other antigen of interest) by these antibodies provides reliable and efficient presentation of the antigen to antibodies contained in test sample, which may be blood, plasma, serum, saliva, etc. Studies using this approach showed exquisite assay specificity, as well as excellent sensitivity and reproducibility.

In general, the disclosed methods of determining exposure of a subject to HDV (or another pathogen or microbe of interest), comprises (i) incubating a liquid biological sample obtained from a subject with a bead particle comprising a core bead coupled to a plurality of antibodies bound to a HDV antigen, such that any antibodies in the liquid biological sample that are capable of binding to the HDV antigen can bind to the decorated surface of the bead particle; (ii) washing the bead particle; (iii) incubating the bead particle with a detectably labeled antibody that binds to an antibody of the species of the subject; and determining the presences or absence of the detectably labeled antibody, wherein the presence of the detectable label indicates the subject was exposed to HDV and the absence of the detectable label indicates the subject was not exposed to HDV.

The subject is a human and another mammal for which it is desired to determine exposure to HDV or another pathogen or microbe of interest. The subject will provide a biological sample, which is generally expected to be a liquid. Suitable sample types include, but are not limited to, blood, plasma, serum, or saliva. In some embodiments, the biological sample may be diluted serum.

The detectably labeled antibody (i.e., secondary antibody) that is incubated with the bead particles to indicate whether the subject possesses antibodies that bind to a HDV antigen. The source of the detectably labeled antibody or secondary antibody is not limited, and may be, for example, human or humanized or may be derived from mouse, rat, sheep, horse, pig, cow, camelid, or other mammal. In some embodiments, detectably labeled antibody or secondary antibody specifically binds to IgG and/or IgM. The detectably labeled antibody or secondary antibody also may bind other types of antibodies as well, so long as the detectably labeled antibody or secondary antibody specifically binds to antibodies from the subject on which the method is being performed. Thus, in some embodiments, the detectably labeled antibody or secondary antibody is an anti-human antibody when the subject on which the method is being performed is a human.

In some embodiments, the detectable label may be selected from among a fluorophore, a pigment (e.g., phycoerythrin), a radioactive isotope, a chemiluminescent molecule, a chromophore, an electron dense label, an enzyme, a dye, a metal, biotin, avidin, streptavidin, and a hapten. However, it should be noted that any detectable label may suffice for the purposes of the disclosed methods.

With respect to the antibody or antibodies that are coupled to the outside of the core bead (i.e., decorated on the core bead), these antibodies may bind directly or indirectly to a HDV antigen or any other antigen of interest. For example, in some embodiments, the antibody may specifically bind to a HDV antigen, while in some embodiments, the antibody may be an anti-histidine antibody (or another tag-specific antibody) and the HDV antigen (or other antigen) may be His-tagged (or comprise another known tag sequence). When a His-tag (or other tag sequence) is desired on the HDV antigen, one can be easily added by producing the HDV antigen recombinately in a bacterial host like E. coli and ensuring that the recombinant sequence encoding the antigen is followed by a sequence encoding histidine repeats (or another tag sequence). Other molecular tags aside from a His-tag (which generally comprises 5-10 histidines) may be used in the present methods as well. For example, other known molecular tag or affinity tags including, but not limited to, chitin binding protein (CBP), maltose binding protein (MBP), Strep-tag (WSHPQFEK), glutathione-S-transferase (GST), thioredoxin (TRX), poly(NANP), FLAG-tag (DYKDDDDK), ALFA-tag, V5-tag, Myc-tag, HA-tag, Spot-tag, T7-tag and NE-tag, ALFA-tag, AviTag, C-tag, calmodulin-tag, polyglutamate tag (e.g., EEEEEE or 5-10 Es), polyarginine tag (e.g., 5-10 Rs), E-tag (GAPVPYPDPLEPR), HA-tag (YPYDVPDYA), Myc-tag (EQKLISEEDL), NE-tag (TKENPRSNQEESYDDNES), S-tag (KETAAAKFERQHMDS), T7-tag (MASMTGGQQMG), Ty-tag (EVHTNQDPLD), V5-tag (GKPIPNPLLGLDST), and Xpress tag (DLYDDDDK). The foregoing and any other peptide tag sequence known in the art may also be suitable for the disclosed methods.

The core bead for the bead particle can also be any bead suitable for multiplex assay platforms like the one described. In particular, polystyrene beads or microparticles are commonly used in this type of assay, and in some embodiments, the core bead may be a polystyrene bead or microparticle. In some embodiments, the polystyrene bead or microparticle may be a LUMINEX® bead or microparticle.

In some embodiments, the amount of the detectable label determined to be present in the liquid biological sample correlates to the amount of anti-HDV IgG and/or IgM in the liquid biological sample.

c. General Methods of Detecting Exposure to a Pathogen or Interest

The method approach described herein can be modified for other analytes or pathogens. In embodiments in which the disclosed bead particles are decorated with anti-His antibodies or other tag-specific antibodies, the only necessary step for altering the antigen specificity is to express a different peptide antigen with a His-tag (or other tag sequence) and contact it with the beads such that the new antigen is presented on the outside surface of the bead. As with the methods involving B. miyamotoi and HDV, when the beads with a different antigen are incubated with a sample (e.g., blood, plasma, mucus, saliva, etc.) from a subject, any antibodies that bind to the selected antigen will bind to the bead and can then be identified by washing the beads and contacting the beads bound with the subject's antibodies with a detectably labeled antibody that specifically binds to antibodies of the subject species.

Accordingly, the methods disclosed herein are not specific solely to B. miyamotoi and HDV, but rather can be used to determine exposure to any number of pathogens or microbes, including but not limited to, viruses, bacteria, fungi, and any other pathogen or microbe that may produce an antibody response in a subject.

### IV. Kits

The present disclosure also provides kits that comprise one or more of the disclosed bead particles and/or kits for use in practicing the disclosed methods. The disclosed kits may comprise, for example, one or more bead particles as disclosed herein and one or more detectably-labeled antibodies that are specific for the subject on which the kit will be used (e.g., anti-human IgG antibodies or anti-human IgM antibodies).

In some embodiments, the disclosed kits comprise a bead particle comprising a microparticle coupled to a plurality of anti-histidine antibodies that are bound to a His-tagged antigen of interest or an antigen or interest comprising another operative tag sequence (e.g., chitin binding protein, maltose binding protein, Strep-tag, glutathione-S-transferase (GST), thioredoxin, poly(NANP), FLAG-tag, ALFA-tag, V5-tag, Myc-tag, HA-tag, Spot-tag, T7-tag and NE-tag, ALFA-tag, AviTag, C-tag, calmodulin-tag, polyglutamate tag, polyarginine tag, E-tag, HA-tag, Myc-tag, NE-tag, S-tag, T7-tag, Ty-tag, V5-tag, or Xpress tag or any other known tag sequence). In some embodiments, the disclosed kits comprise a bead particle comprising a microparticle coupled to a plurality of antibodies bound to a B. miyamotoi glycerophosphodiester phosphodiesterase (glpQ), either directly or indirectly via a tag sequence. In some embodiments, the disclosed kits comprise a bead particle comprising a microparticle coupled to a plurality of antibodies bound to a HDV antigen, either directly or indirectly via a tag sequence. In some embodiments, the microparticle may be a polystyrene bead (e.g., a LUMINEX® bead). Any bead or bead particle disclosed herein may be included in a kit.

In some embodiments, the disclosed kits may further comprise one or more detectably labeled antibod(ies). The detectably labeled antibodies must be able to recognize or bind to antibodies from the species from which an assayed sample is derived. For instance, if the kit is to be used to assess whether a human has been exposed to B. miyamotoi (i.e., to detect the presence or absence of antibodies that bind to B. miyamotoi glpQ), then the detectably labeled antibodies must bind to human antibodies. In other words, the one or more detectably labeled antibodies may be one or more anti-human antibodies. In some embodiments, the detectably labeled antibody (or antibodies) may specifically bind to IgG and/or IgM. In some embodiments, the detectably labeled antibody may bind to human IgG and/or IgM. In some embodiments, the detectably labeled antibody may bind to human IgG. In some embodiments, the detectably labeled antibody may bind to human IgM.

The detectable label attached to the one or more detectably labeled antibodies is not particularly limited and may be selected from among a fluorophore, a pigment (e.g., phycoerythrin), a radioactive isotope, a chemiluminescent molecule, a chromophore, an electron dense label, an enzyme, a dye, a metal, biotin, avidin, streptavidin, and a hapten.

Typically, the disclosed kits will also include instructions recorded in a tangible form (e.g., contained on paper or an electronic medium) for using the packaged materials (e.g., bead particles, detectably labeled antibodies, etc.) for determining the presence or amount of antibodies that bind to a pathogen or microbe of interest (e.g., B. miyamotoi, HDV, or any other virus, bacteria, or fungi) in a test sample.

The various components of the disclosed kits or systems may be provided in a variety of forms. For example, in some embodiments, any included enzymes, probes, and/or antibodies may be provided in a lyophilized form. Such lyophilized reagents may be pre-mixed before lyophilization so that when reconstituted they form a complete mixture with the proper ratio of each of the components ready for use in the assay. Alternatively, such lyophilized reagents may be provided separately, and thus may require mixing once reconstituted for use in the assay. In addition, the kits may contain a reconstitution reagent for reconstituting the lyophilized reagents of the kit. However, in some embodiments, any included enzymes, probes, and/or antibodies may be provided in a liquid form.

The following examples are given to illustrate the present invention. It should be understood, however, that the invention is not to be limited to the specific conditions or details described in these examples.

### EXAMPLES

### Example 1—Validation of Methods for Detecting Exposure to B. miyamotoi

In this bead-based immunoassay (IA), anti-Histidine (His) monoclonal antibodies were coupled to magnetic carboxylated microspheres using a two-step carbodiimide reaction. The target antigen, His-labeled B. miyamotoi glycerophosphodiester phosphodiesterase (glpQ) was loaded onto the coupled microspheres in a subsequent incubation. In the test setup, monoclonal antibody-coupled beads with and without captured antigen were mixed to form a duplex (each beadset assigned a respective Luminex spectral address). Duplicate wells (one each for IgG and IgM) containing these duplexes were incubated with each serum sample in 96-well microplates. Sample B. miyamotoi IgG and/or IgM bound to the captured glpQ antigen, while the uncaptured beads served as an internal sample control. Following the serum incubation and washing, phycoerythrin-conjugated anti-human IgG and/or IgM was added to assigned wells. After the conjugate incubation and washing, the microspheres were analyzed on the Luminex FlexMap 3D instrument. The fluorescence generated was normalized by each internal sample control bead and the net fluorescence was proportional to the amount of B. miyamotoi glpQ IgG or IgM present in each serum sample. The raw median fluorescence intensity (MFI) generated by each specimen was converted into an index value relative to a cutoff calibrator serum included with each assay plate.

### Reagents and Instruments

TABLE 1
Reagents and uses

| Reagent: | Use: |
|---|---|
| Luminex Magplex | Capture monoclonal antibody |
| carboxylated microspheres | coupling substrate |
| Mouse anti-His | Capture monoclonal antibodies |
| B. miyamotoi glpQ | Captured Antigen |
| Phycoerythrin-conjugated | Fluorescent IgG detector antibody |
| anti-human IgG | |
| Phycoerythrin-conjugated | Fluorescent IgM detector antibody |
| anti-human IgM | |

Instrument used-FlexMap 3D; S/N FM3DD19021021
Specimen type—Serum

### Precision

Within-run precision or intra-assay variation was assessed using a panel of 3 samples where 8 wells were tested on the same assay setup. The resultant index values are shown in the table below:

TABLE 2
IgG Intra-assay variation

| | Sample 656318 | | Sample 967188 | | Sample 976370 | |
|---|---|---|---|---|---|---|
| Test | IgG | | IgG | | IgG | |
| well | Index | Interpret. | Index | Interpret. | Index | Interpret. |
| 1 | 1.31 | Positive | 0.26 | Negative | 0.12 | Negative |
| 2 | 1.29 | Positive | 0.24 | Negative | 0.11 | Negative |
| 3 | 1.30 | Positive | 0.25 | Negative | 0.11 | Negative |
| 4 | 1.31 | Positive | 0.24 | Negative | 0.11 | Negative |
| 5 | 1.23 | Positive | 0.24 | Negative | 0.11 | Negative |
| 6 | 1.23 | Positive | 0.25 | Negative | 0.11 | Negative |
| 7 | 1.29 | Positive | 0.25 | Negative | 0.11 | Negative |
| 8 | 1.27 | Positive | 0.26 | Negative | 0.11 | Negative |
| mean | 1.28 | | 0.25 | | 0.11 | |
| SD | 0.03 | | 0.01 | | 0.003 | |
| % CV | 3% | | 3% | | 3% | |

TABLE 3
IgM Intra-assay variation

| | Sample 656318 | | Sample 967188 | | Sample 976370 | |
|---|---|---|---|---|---|---|
| Test | IgM | | IgM | | IgM | |
| well | Index | Interpret. | Index | Interpret. | Index | Interpret. |
| 1 | 2.26 | Positive | 0.09 | Negative | 0.14 | Negative |
| 2 | 2.41 | Positive | 0.09 | Negative | 0.14 | Negative |
| 3 | 2.23 | Positive | 0.09 | Negative | 0.13 | Negative |
| 4 | 2.32 | Positive | 0.09 | Negative | 0.14 | Negative |
| 5 | 2.26 | Positive | 0.09 | Negative | 0.13 | Negative |
| 6 | 2.16 | Positive | 0.09 | Negative | 0.13 | Negative |
| 7 | 2.71 | Positive | 0.09 | Negative | 0.14 | Negative |
| 8 | 2.57 | Positive | 0.09 | Negative | 0.15 | Negative |
| mean | 2.37 | | 0.09 | | 0.14 | |
| SD | 0.19 | | 0.003 | | 0.01 | |
| % CV | 8% | | 3% | | 5% | |

For all within-run results obtained, the qualitative concordance was 100% and the % CV for all positive values was <8%. This met the acceptance criteria of 100% qualitative concordance, and % CV<15% for the positive specimen.

These same samples were run on 5 different setup dates. The values obtained for each sample are shown in the table below:

TABLE 4
IgG Inter-assay variation

| | Sample 656318 | | Sample 967188 | | Sample 976370 | |
|---|---|---|---|---|---|---|
| Test | IgG | | IgG | | IgG | |
| run | Index | Interpret. | Index | Interpret. | Index | Interpret. |
| 1 | 1.52 | Positive | 0.31 | Negative | 0.14 | Negative |
| 2 | 1.41 | Positive | 0.25 | Negative | 0.12 | Negative |
| 3 | 1.38 | Positive | 0.24 | Negative | 0.11 | Negative |
| 4 | 1.28 | Positive | 0.25 | Negative | 0.11 | Negative |
| 5 | 1.27 | Positive | 0.25 | Negative | 0.11 | Negative |
| mean | 1.37 | | 0.26 | | 0.12 | |
| SD | 0.10 | | 0.03 | | 0.01 | |
| % CV | 8% | | 11% | | 11% | |

TABLE 5
IgM Inter-assay variation

| | Sample 656318 | | Sample 967188 | | Sample 976370 | |
|---|---|---|---|---|---|---|
| Test | IgM | | IgM | | IgM | |
| run | Index | Interpret. | Index | Interpret. | Index | Interpret. |
| 1 | 2.62 | Positive | 0.13 | Negative | 0.18 | Negative |
| 2 | 2.39 | Positive | 0.10 | Negative | 0.16 | Negative |
| 3 | 2.40 | Positive | 0.10 | Negative | 0.15 | Negative |
| 4 | 2.37 | Positive | 0.09 | Negative | 0.14 | Negative |
| 5 | 2.48 | Positive | 0.10 | Negative | 0.13 | Negative |
| mean | 2.45 | | 0.10 | | 0.15 | |
| SD | 0.10 | | 0.01 | | 0.02 | |
| % CV | 4% | | 13% | | 12% | |

For all sample results the qualitative concordance was 100% and the % CV for all positive values (Index≥1.0) was ≤8%. This met the acceptance criteria of 100% qualitative concordance, and % CV≤20% for the positive specimen.

### Analytical Specificity

Cross-reactivity: A panel of 66 samples positive for antibodies to other tick-borne organisms (HGA, HME, Lyme, B. hermsii, B. microti) and spirochetes (Treponema pallidum) was tested for B. miyamotoi IgG and IgM with the following findings. The results are shown below:

TABLE 6

| | IgG | IgM |
|---|---|---|
| No. negative: | 60 | 63 |
| % negative: | 91% | 95% |

The breakdown of glpQ IgG and/or IgM positivity is summarized below:

TABLE 7

| Antibody | | glpQ | | glpQ | |
|---|---|---|---|---|---|
| positivity | N | IgG+ | Index | IgM+ | Index |
| B. microti | 20 | 2 | ≤2.2 | 1 | 1.6 |
| HGA/HME | 15 | 1 | 1.1 | 2 | ≤2.6 |
| T. pallidum | 15 | 3 | ≤2.5 | 0 | n/a |
| B. hermsii | 4 | 0 | n/a | 0 | n/a |
| B. burgorferi | 12 | 0 | n/a | 0 | n/a |

These results met the acceptance criteria of > or =80% of samples testing negative for glpQ IgG and > or =90% of samples testing negative for glpQ IgM.

False positive rate: The false positive rate was assessed by the results obtained on the panel of 60 blood donor samples used to set the assay cutoff (samples were drawn in 2017 from San Francisco Bay Area and Arizona blood donors). Since the 95^{th} percentile index values for this group were used to discriminate between positive and negative IgG and IgM interpretations, the expected false positive rate is approximately 5% for both glpQ IgG and IgM.

For the 4 glpQ IgG+ samples in this group: 3 showed index values between 1.0-1.1 and 1 showed an index value of 5.2. For the 4 glpQ IgM+ samples in this group: 3 showed index values between 1.0-1.3 and 1 showed an index value of 16.3.

### Accuracy

Method comparison study: A panel of 56 samples with previous results for glpQ IgG and IgM was used to perform method comparison studies. These specimens were either obtained from Imugen, Inc. (a division of Oxford Immunotech) or provided by Focus Diagnostics, Inc. and further tested for glpQ IgG and IgM by Yale School of Public Health in 2013. The methodology used as the reference method was ELISA (Imugen) or ELISA with positive reflex to Western Blot (Yale). Results \summarized below:

TABLE 8

| | N = 56 | |
|---|---|---|
| | IgG | IgM |
| No. concordant: | 46 | 40 |
| % concordance: | 82% | 71% |

Although the overall IgG concordance met the acceptance criteria of > or =80%, the IgM concordance of 71% represents a deviation. Further analysis of results showed good negative percent agreement (>90%) with both reference methods (Imugen and Yale). The results compared to Imugen's results for the IgM-positive group suggested that specificity issues may be contributing to the observed assay discordance.

In order to compare assay specificities, 15 samples positive for antibodies to HME or HGA (among those used for the specificity studies described above) were sent to Imugen for testing in their IgG and IgM ELISA's. The comparison of findings by the 2 laboratories for this HME/HGA panel is shown below:

TABLE 9

| glpQ | | glpQ | glpQ | Total | Indeter- |
|---|---|---|---|---|---|
| IgG/M test | N | IgG+ | IgM+ | antibody+ | minate |
| Imugen | 15 | 3 (20%) | 4 (27%) | 7 (47%) | 4 (27%) |
| (ELISA) | | | | | |
| QDID (IA) | 15 | 1 (7%) | 2 (13%) | 3 (20%) | 0 (0%) |

The number of positive samples in this group when tested at Imugen in their assays was notably higher than that found by QDID. Although co-infection with B. miyamotoi and other tick-borne organisms is possible, it seems dubious to ascribe coinfection or even exposure to so many HME/HGA antibody-positive samples that are also positive for glpQ antibodies by ELISA. Moreover, it is interesting that 4/15 (27%) of these samples were indeterminate by Imugen's ELISA's due to background reactivity. It is therefore reasonable to suspect that some of the Imugen results in the method comparison may not represent true positives, reducing between-assay positive percent agreement and in turn reducing overall concordance.

Concordance was further evaluated by omitting from analysis selected sample results where the observed inter-assay variation includes an interpretation crossover (i.e., borderline results). The table below shows the following criteria for exclusion from the method comparison panel (including one sample with high intra-well background):

TABLE 10

| Discordant pattern: | | | |
|---|---|---|---|
| Reference | QDID | | |
| method: | IA: | # | Criteria: |
| + (Imugen) | − | IgG: 2 | Imugen index not greater |
| | | IgM: 2 | than 20% above 1.0 cutoff |
| + (Imugen) | − | IgM: 2 | QDID negative index not greater |
| | | | than 13% below 1.0 cutoff |
| − (Yale) | + | IgM: 1 | QDID positive index not greater |
| | | | than 12% above 1.0 cutoff |
| + (Imugen) | − | IgM: 1 | High QDID sample control background |

The table below shows the reanalyzed concordance results for the adjusted sample population:

TABLE 11

| | IgG | IgM |
|---|---|---|
| | (N = 54) | (N = 50) |
| No. concordant: | 46 | 40 |
| % concordance: | 85% | 80% |

As expected, the overall concordance was increased for both IgG and IgM. These findings meet the stated criteria of at least 80% overall concordance with the combined reference methods.

### Clinical Sensitivity and Specificity

Per a study in 2013-2014 of serologic glpQ results for 97 confirmed cases of B. miyamotoi disease, serologic testing was found to be less sensitive in identifying patients with acute infection than those in convalescence (16% vs. 78% positivity, respectively). Therefore, it is recommended that PCR be performed in suspected cases of acute B. miyamotoi infection. However, the spirochetemic window is narrow in acute infection; therefore, if B. miyamotoi is initially undetected by PCR, a positive antibody test on a later convalescing specimen may support a diagnosis of B. miyamotoi disease. (Molloy P J, et al, Ann Intern Med. 2015; 163:91-98. doi: 10.7326/M15-0333.)

Clinical specificity of antibody detection is dependent upon glpQ gene homology; although glpQ is present in other relapsing fever borreliae, the degree of sequence proximity determines the expected likelihood of antibody crossreactivity. The glpQ sequence found in B. lonestari is most similar to B. miyamotoi (85% identical) and to lesser degrees similar to glpQ in B. hermsii (83.4%), B. parkeri (81.9%), B. turicatae (81.3%), B. coriaceae (79.5%), B. crocidurae (76.9%) and B. recurrentis (76.6%). The organisms causing Lyme disease, human granulocytic anaplasmosis, and Powassan virus disease lack the glpQ protein and are therefore not expected to cross react with B. miyamotoi glpQ. (Bacon, R M, J of Clin Micro 2004; 42:2326-2328).

### Reference Range Verification

A panel of 58 blood donor samples obtained from non-endemic regions (other than specimens used to establish reference ranges) were tested for glpQ IgG and IgM. The results are summarized in the table below:

TABLE 12

| | IgG | IgM |
|---|---|---|
| No. negative: | 57 | 58 |
| % negative: | 98% | 100% |

These results met acceptance criteria of ≥90% and ≥95% seronegativity for IgG and IgM, respectively.

### Specimen Stability

Triplicate wells for 5 samples (3 positive, 2 negative) were tested to evaluate sample stability. Samples were stored 7 days at room temperature, refrigerated 14 days, frozen 30 days (−20° C. and −70° C.), and also subjected to 3 freeze/thaw cycles (−20° C. and −70° C.).

The results of these studies met acceptance criteria of 100% qualitative concordance with < or =20% CV with the respective initial result.

Further stability studies were performed where samples were tested after frozen storage for at least 80 days (89 days at −70° C. and 84 days at −20 C). The results are shown in Tables 8a-8b of the Appendix and are summarized below:
All results showed 100% qualitative agreement with the initial results
The CVs for all positive values but one were < or =20% CV with the initial results
For one positive IgM sample, the CV for results obtained before and after storage at −20 C was 27%. However, the stored index was higher than the initial value, and the % CV for the stored result and that of the mean sample inter-assay variation was only 7%. Therefore, the 27% CV finding is not considered a deviation.
One negative sample gave a stored IgG index of 22% CV at −70 C and 25% at −20 C. Since both the initial and stored indexes were very low (<0.20), this variation is not considered a deviation; furthermore % CV's for negative values are generally not required in semi-quantitative assay validations.
One positive sample was not included in −20 C extended frozen studies due to insufficient volume. Nonetheless, the remaining 4 panel samples were more than required per QPLN.110.00452.

The above findings indicated that samples stored frozen for 80 days are acceptable for B. miyamotoi IgG and IgM testing.

Based on these results, it was determined:
Room Stability (18.0 to 26.0° C.): 7 days
Refrigerated Stability (2.0 to 8.0° C.): 14 days
Frozen Stability (−10.0 to −80.0° C.): 80 days
Freeze/Thaw Stability: 3 cycles

### Interfering Substances

To assess interference, samples (2 positives, 1 negative) were spiked with slight and moderate levels of hemolysin. The % CV's of these results with those obtained from the non-hemolyzed samples are shown below:

| | | IgG % CV | | IgM % CV | |
|---|---|---|---|---|---|
| | | Slight | Moderate | Slight | Moderate |
| Sample | Result | Hemolysis | Hemolysis | Hemolysis | Hemolysis |
| 656318 | Positive | 4% | 2% | 1% | 1% |
| 609946 | Positive | 1% | 1% | 1% | 3% |
| 967188 | Negative | 2% | 1% | 4% | 8% |

The index values and interpretations for each sample were determined. These results met acceptance criteria of 100% qualitative concordance and index CV's< or =20% between the non-hemolyzed and hemolyzed index values, respectively.

### Conclusion

This validation study has been reviewed and the performance of the method is considered acceptable for patient testing. Data for this study supports the clinical use of this assay.

### Example 2—Validation of Methods for Detecting Exposure to Hepatitis D Virus

In this bead-based immunoassay (IA), anti-Histidine (His) monoclonal antibodies are coupled to magnetic carboxylated microspheres using a two-step carbodiimide reaction. The target antigen, His-labeled HDV antigen, is loaded onto the coupled microspheres in a subsequent incubation. In the test setup, monoclonal antibody-coupled beads with and without captured antigen are mixed to form a duplex (each beadset assigned a respective Luminex spectral address). Wells containing these duplexes are incubated with each serum sample in 96-well microplates. Sample HDV antibodies bind to the captured antigen, while the uncaptured beads serve as an internal sample control. Following the serum incubation and washing, phycoerythrin-conjugated anti-human IgM is added to the wells. After the conjugate incubation and washing, the microspheres are analyzed on the Luminex FlexMap 3D instrument. The fluorescence generated is normalized by each internal sample control bead and the net fluorescence is proportional to the amount of HDV IgM present in each serum. The raw median fluorescence intensity (MFI) generated by each specimen is converted into an index value relative to a cutoff calibrator serum included with each assay plate. Index values <1.0 were interpreted as negative; > or =1.0, as positive.

### Reagents and Instruments

TABLE 13
Reagents and uses

| Reagent: | Use: |
|---|---|
| Luminex Magplex | Capture monoclonal antibody |
| carboxylated microspheres | coupling substrate |
| Mouse anti-His | Capture monoclonal antibodies |
| Hepatitis D (HDV) | Captured Antigen |
| recombinant antigen | |
| Phycoerythrin-conjugated | Fluorescent IgM detector antibody |
| anti-human IgM | |

Instrument used-FlexMap 3D; S/N FM3DD19021021
Specimen type—Serum

### Precision

Within-run precision or intra-assay variation was assessed using a panel of 4 samples where 8 wells were tested on the same assay setup. The resultant index values are shown in the table below:

TABLE 14
Intra-assay variation

| | C75611767 | | C87617173 | | D16985993 | | D17021937 | |
|---|---|---|---|---|---|---|---|---|
| | IgM | | IgM | | IgM | | IgM | |
| Test well | Index | Interpret | Index | Interpret | Index | Interpret | Index | Interpret |
| 1 | 1.00 | Positive | 1.60 | Positive | 0.14 | Negative | 0.15 | Negative |
| 2 | 0.93 | Repeat | 1.51 | Positive | 0.14 | Negative | 0.14 | Negative |
| 3 | 1.03 | Positive | 1.52 | Positive | 0.14 | Negative | 0.14 | Negative |
| 4 | 1.27 | Positive | 1.52 | Positive | 0.13 | Negative | 0.13 | Negative |
| 5 | 1.13 | Positive | 1.55 | Positive | 0.14 | Negative | 0.15 | Negative |
| 6 | 1.05 | Positive | 1.52 | Positive | 0.14 | Negative | 0.15 | Negative |
| 7 | 1.02 | Positive | 1.61 | Positive | 0.15 | Negative | 0.14 | Negative |
| 8 | 1.02 | Positive | 1.57 | Positive | 0.14 | Negative | 0.15 | Negative |

| | | | | |
|---|---|---|---|---|
| mean | 1.05 | 1.55 | 0.14 | 0.14 |
| SD | 0.10 | 0.04 | n/a | n/a |
| % CV | 10% | 3% | n/a | n/a |

For all within-run results obtained the % CV for all positive values was ≤10%, and 100% qualitative agreement was found for all but one well for C75611767, a borderline sample. Although the 7 other replicates showed 100% qualitative agreement as positive, borderline samples (initial indexes 0.8-1.2) were repeated in duplicate. Therefore, shown below are the results for the repeated duplicate wells:

TABLE 15

| | Index | Interpretation |
|---|---|---|
| Well 1 | 1.19 | Positive |
| Well 2 | 1.32 | Positive |
| mean | 1.25 | Positive |

These results met the acceptance criteria of 100% qualitative concordance, and % CV≤15% for positive specimens.

These same samples were run on 5 different setup dates. The values obtained for each sample are shown in the following table:

TABLE 16
Inter-assay variation

| | C75611767 | | C87617173 | | D16985993 | | D17021937 | |
|---|---|---|---|---|---|---|---|---|
| | IgM | | IgM | | IgM | | IgM | |
| Test run | Index | Interpret. | Index | Interpret. | Index | Interpret. | Index | Interpret. |
| 1 | 1.12 | Positive | 1.94 | Positive | 0.17 | Negative | 0.15 | Negative |
| 2 | 1.40 | Positive | 2.06 | Positive | 0.15 | Negative | 0.14 | Negative |
| 3 | 1.36 | Positive | 1.73 | Positive | 0.15 | Negative | 0.16 | Negative |
| 4 | 1.19 | Positive | 1.91 | Positive | 0.14 | Negative | 0.11 | Negative |
| 5 | 1.28 | Positive | 1.92 | Positive | 0.16 | Negative | 0.17 | Negative |

| | | | | |
|---|---|---|---|---|
| mean | 1.27 | 1.91 | 0.15 | 0.15 |
| SD | 0.12 | 0.12 | n/a | n/a |
| % CV | 9% | 6% | n/a | n/a |

For all sample results the qualitative concordance was 100% and the % CV for all positive values (Result≥1.0) was ≤9%. This met the acceptance criteria of 100% qualitative concordance, and % CV≤20% for positive specimens.

### Analytical Specificity

Cross-reactivity: A panel of 40 samples positive for antibodies to Hepatitis A, C, or E, and negative for Hepatitis B surface antigen and core antibody, was tested for HDV total antibodies with the following findings:

TABLE 17
N = 40

| | |
|---|---|
| No. Negative: | 40 |
| % Negative | 100% |

These results met the acceptance criteria of > or =80% of samples testing negative for HDV IgM.

Blood donor serology: A panel of 43 blood donor samples was used to determine the cutoff for IgM positivity. Hepatitis B screening is routinely performed on donors (with positivity criteria for exclusion); therefore all samples in this panel are presumed negative for HBV infection and hence represent an HDV naïve population that should also be negative for all of its markers in serum, including IgM antibodies. The negative indexes for this group were calculated using the determined cutoff. This cutoff was verified by testing an additional 40 blood donor samples.

False Positive Rate: The false positive rate was assessed by the collective findings for 40 cross-reactivity and 83 blood donor samples. Since 100% of tested samples in these groups were negative for IgM antibodies, the false positive rate is expected to be <1%.

### Accuracy

Method comparison study: A panel of 52 samples with previous results for HDV IgM was used to perform method comparison studies. The overall concordance is shown in the table below:

TABLE 18
N = 52

| | |
|---|---|
| # concordant: | 48 |
| % concordance: | 92% |

A breakdown of the 4 discordant samples is shown in the following table:

TABLE 19

| Discordant pattern: | | | |
|---|---|---|---|
| Reference | | | |
| method (EIA): | IgM IA: | # | Note: |
| Positive | Negative | 3 | N = 2: Total antibody |
| | | | positive |
| | | | N = 1: Total antibody |
| Negative | Positive | 1 | Total antibody positive |

These results met acceptance criteria of 80% overall concordance with the current assay for HDV IgM.

### Clinical Sensitivity and Specificity

Sensitivity: A subset of 21 samples used for method comparison also tested positive for HDV RNA. The results in are summarized below:

TABLE 20

| N = 21 | Luminex IA IgM+ | EIA IgM+ |
|---|---|---|
| # positive: | 7 | 6 |
| % positive: | 33% | 29% |

As also shown above, comparable levels of positivity were found between the IA and EIA assays, respectively, with most samples testing IgM negative in this group. Limited data are available for the expected IgM levels in HDV RNA+ patients; even so, all of these samples tested positive for HDV total antibodies.

Specificity: 83/83 (100%) of blood donor samples (pre-screened for HBV markers) tested negative for HDV IgM antibodies. In addition, the 40 samples positive for other hepatitis viruses that were used for cross-reactivity studies also tested negative for HBV surface antigen and core antibody; all 40 samples (100%) were found negative for HDV IgM antibodies.

Reference range verification: To verify the reference range, a 2^{nd} panel of 40 additional blood donor samples was tested for HDV IgM antibodies. The results are summarized below:

TABLE 21
N = 40

| | |
|---|---|
| # negative: | 40 |
| % negative: | 100% |

These results met acceptance criteria of 100% seronegativity for HDV IgM antibodies.

### Specimen Stability

A panel of 4 samples (2 positives, 2 negatives) was tested to evaluate sample stability. Samples were stored 7 days at room temperature, refrigerated 14 days, frozen 30 days (−20° C. and −70° C.), and subjected to 3 freeze/thaw cycles (−20° C. and −70° C.). These results met acceptance criteria of 100% qualitative concordance with < or =20% CV with the respective initial result.

Based on these results, it was determined:
Room Stability (18.0 to 26.0° C.): 7 days
Refrigerated Stability (2.0 to 8.0° C.): 14 days
Frozen Stability (−10.0 to −80.0° C.): 30 days
Freeze/Thaw Stability: 3 cycles

### Interfering Substances

To assess interference, 2 samples (1 positive, 1 negative) were spiked with slight and moderate levels of hemolysis The results compared with those obtained from the non-hemolyzed samples are shown below:

TABLE 22

| | IgM Result | | | Index % CV | |
|---|---|---|---|---|---|
| | No | Slight | Moderate | Slight | Moderate |
| Sample | Hemolysis | Hemolysis | Hemolysis | Hemolysis | Hemolysis |
| C87617173 | Positive | Positive | Positive | 9% | 10% |
| D16985993 | Negative | Negative | Negative | n/a | n/a |

These results met acceptance criteria of 100% qualitative concordance between the non-hemolyzed and hemolyzed index values, respectively.

### Conclusion

This validation study has been reviewed and the performance of the method is considered acceptable for patient testing. Data for this study supports the clinical use of this assay.

### Example 3-Validation of Further Methods for Detecting Exposure to Hepatitis D Virus

In this bead-based immunoassay (IA), anti-Histidine (His) monoclonal antibodies are coupled to magnetic carboxylated microspheres using a two-step carbodiimide reaction. The target antigen, His-labeled HDV antigen, is loaded onto the coupled microspheres in a subsequent incubation. In the test setup, monoclonal antibody-coupled beads with and without captured antigen are mixed to form a duplex (each beadset assigned a respective Luminex spectral address). Wells containing these duplexes are incubated with each serum sample in 96-well microplates. Sample HDV antibodies bind to the captured antigen, while the uncaptured beads serve as an internal sample control. Following the serum incubation and washing, a cocktail containing phycoerythrin-conjugated anti-human IgG, IgA and IgM is added to the wells. After the conjugate incubation and washing, the microspheres are analyzed on the Luminex FlexMap 3D instrument. The fluorescence generated is normalized by each internal sample control bead and the net fluorescence is proportional to the amount of HDV antibodies present in each serum. The raw median fluorescence intensity (MFI) generated by each specimen is converted into an index value relative to a cutoff calibrator serum included with each assay plate. Index values <1.0 were interpreted as negative; > or =1.0, as positive.

### Reagents and Instruments

TABLE 23
Reagents and uses

| Reagent: | Use: |
|---|---|
| Luminex Magplex carboxylated | Capture monoclonal antibody |
| microspheres | coupling substrate |
| Mouse anti-His | Capture monoclonal antibodies |
| Hepatitis D (HDV) recombinant | Captured Antigen |
| antigen | |
| Phycoerythrin-conjugated | Fluorescent detector cocktail |
| anti-human IgG | components |
| Phycoerythrin-conjugated | |
| anti-human IgA | |
| Phycoerythrin-conjugated | |
| anti-human IgM | |

Instrument used—FlexMap 3D; S/N FM3DD19021021
Specimen type—Serum

### Precision

Within-run precision or intra-assay variation was assessed using a panel of 4 samples where 8 wells were tested on the same assay setup. The resultant index values are shown in the table below:

TABLE 24
Intra-assay variation

| | C75611767 | | C87617173 | | D16985993 | | D17021937 | |
|---|---|---|---|---|---|---|---|---|
| Test | Total | | Total | | Total | | Total | |
| well | Ig Index | Interpret | Ig Index | Interpret | Ig Index | Interpret | Ig Index | Interpret |
| 1 | 6.49 | Positive | 3.59 | Positive | 0.16 | Negative | 0.18 | Negative |
| 2 | 6.32 | Positive | 3.53 | Positive | 0.19 | Negative | 0.17 | Negative |
| 3 | 6.31 | Positive | 3.49 | Positive | 0.17 | Negative | 0.18 | Negative |
| 4 | 5.94 | Positive | 3.38 | Positive | 0.17 | Negative | 0.19 | Negative |
| 5 | 6.45 | Positive | 3.42 | Positive | 0.17 | Negative | 0.18 | Negative |
| 6 | 6.69 | Positive | 3.53 | Positive | 0.19 | Negative | 0.17 | Negative |
| 7 | 6.46 | Positive | 3.14 | Positive | 0.19 | Negative | 0.17 | Negative |
| 8 | 5.75 | Positive | 3.45 | Positive | 0.18 | Negative | 0.18 | Negative |

| | | | | |
|---|---|---|---|---|
| mean | 6.30 | 3.44 | 0.18 | 0.18 |
| SD | 0.31 | 0.14 | n/a | n/a |
| % CV | 5% | 4% | n/a | n/a |

For all within-run results obtained, the qualitative concordance was 100% and the % CV for all positive values was ≤5%. This met the acceptance criteria of 100% qualitative concordance, and % CV≤15% for positive specimens.

These same samples were run on 5 different setup dates. The values obtained for each sample are shown in the following table:

TABLE 25
Inter-assay variation

| | C75611767 | | C87617173 | | D16985993 | | D17021937 | |
|---|---|---|---|---|---|---|---|---|
| Test | Total | | Total | | Total | | Total | |
| run | Ig Index | Interpret. | Ig Index | Interpret. | Ig Index | Interpret. | Ig Index | Interpret. |
| 1 | 6.42 | Positive | 3.96 | Positive | 0.20 | Negative | 0.18 | Negative |
| 2 | 5.81 | Positive | 4.33 | Positive | 0.25 | Negative | 0.16 | Negative |
| 3 | 6.70 | Positive | 4.53 | Positive | 0.20 | Negative | 0.16 | Negative |
| 4 | 6.52 | Positive | 4.84 | Positive | 0.32 | Negative | 0.18 | Negative |
| 5 | 6.34 | Positive | 4.54 | Positive | 0.22 | Negative | 0.18 | Negative |

| | | | | |
|---|---|---|---|---|
| mean | 6.36 | 4.44 | 0.24 | 0.17 |
| SD | 0.33 | 0.32 | n/a | n/a |
| % CV | 5% | 7% | n/a | n/a |

For all sample results the qualitative concordance was 100% and the % CV for all positive values (Result≥1.0) was ≤7%. This met the acceptance criteria of 100% qualitative concordance, and % CV≤20% for positive specimens.

### Analytical Specificity

Cross-reactivity: A panel of 40 samples positive for antibodies to Hepatitis A, C, or E, and negative for Hepatitis B surface antigen and core antibody, was tested for HDV total antibodies with the following findings:

TABLE 26
N = 40

| | |
|---|---|
| No. Negative: | 40 |
| % Negative | 100% |

These results met the acceptance criteria of > or =80% of samples testing negative for HDV total antibodies.

Blood donor serology: A panel of 43 blood donor samples was used to determine the cutoff for IgM positivity. Hepatitis B screening is routinely performed on donors (with positivity criteria for exclusion); therefore all samples in this panel are presumed negative for HBV infection and hence represent an HDV naïve population that should also be negative for all of its markers in serum, including total antibodies. The negative indexes for this group were calculated using the determined cutoff. This cutoff was verified by testing an additional 40 blood donor samples.

False Positive Rate: The false positive rate was assessed by the collective findings for 40 cross-reactivity and 83 blood donor samples. Since 100% of tested samples in these groups were negative for total antibodies, the false positive rate is expected to be <1%.

### Accuracy

M A panel of 62 samples with previous results for HDV total antibodies was used to perform method comparison studies. The overall concordance is shown in the table below:

TABLE 27
N = 62

| | |
|---|---|
| # concordant: | 59 |
| % concordance: | 95% |

A breakdown of the 3 discordant samples is shown in the following table:

TABLE 28

| Discordant pattern: | | | |
|---|---|---|---|
| Reference | | | |
| method (EIA): | Ig IA: | # | Note: |
| Positive | Negative | 2 | Both HDV IgM negative |
| Negative | Positive | 1 | IA index >4; IgM- |
| | | | negative, HDV RNA+ |

These results met acceptance criteria of 80% overall concordance with the current assay for HDV total antibodies.

### Clinical Sensitivity and Specificity

Sensitivity: A subset of 30 samples used for method comparison also tested positive for HDV RNA. The results in are summarized below:

TABLE 29
N = 30

| | |
|---|---|
| # positive: | 30 |
| % positive: | 100% |

Although the EIA showed excellent sensitivity based on the positivity in this group (29/30, 97%), it fell slightly below the 100% value obtained for HDV RNA+ samples tested by IA.

Specificity: 83/83 (100%) of blood donor samples (pre-screened for HBV markers) tested negative for HDV total antibodies. In addition, the 40 samples positive for other hepatitis viruses that were used for cross-reactivity studies also tested negative for HBV surface antigen and core antibody; all 40 samples (100%) were found negative for HDV total antibodies.

Reference range verification: To verify the reference range, a 2^{nd} panel of 40 additional blood donor samples was tested for HDV total antibodies. The results are summarized below:

TABLE 30
N = 40

| | |
|---|---|
| # negative: | 40 |
| % negative: | 100% |

These results met acceptance criteria of 100% seronegativity for HDV total antibodies.

### Specimen Stability

A panel of 4 samples (2 positives, 2 negatives) was tested to evaluate sample stability. Samples were stored 7 days at room temperature, refrigerated 14 days, frozen 30 days (−20° C. and −70° C.), and subjected to 3 freeze/thaw cycles (−20° C. and −70° C.). These results met acceptance criteria of 100% qualitative concordance with < or =20% CV with the respective initial result.

Based on these results, it was determined:
Room Stability (18.0 to 26.0° C.): 7 days
Refrigerated Stability (2.0 to 8.0° C.): 14 days
Frozen Stability (−10.0 to −80.0° C.): 30 days
Freeze/Thaw Stability: 3 cycles

### Interfering Substances

To assess interference, 2 samples (1 positive, 1 negative) were spiked with slight and moderate levels of hemolysis The results compared with those obtained from the non-hemolyzed samples are shown below:

TABLE 31

| | Total Antibody Result | | | Index % CV | |
|---|---|---|---|---|---|
| | No | Slight | Moderate | Slight | Moderate |
| Sample | Hemolysis | Hemolysis | Hemolysis | Hemolysis | Hemolysis |
| C87617173 | Positive | Positive | Positive | 5% | 1% |
| D16985993 | Negative | Negative | Negative | 1% | 9% |

These results met acceptance criteria of 100% qualitative concordance between the non-hemolyzed and hemolyzed index values, respectively.

### Conclusion

This validation study has been reviewed and the performance of the method is considered acceptable for patient testing. Data for this study supports the clinical use of this assay.

All patents and publications mentioned in the specification are indicative of the levels of those of ordinary skill in the art to which the disclosure pertains. All patents and publications are herein incorporated by reference to the same extent as if each individual publication was specifically and individually indicated to be incorporated by reference.

Further, one skilled in the art readily appreciates that the present disclosure is well adapted to carry out the objects and obtain the ends and advantages mentioned, as well as those inherent therein. Modifications therein and other uses will occur to those skilled in the art. These modifications are encompassed within the spirit of the disclosure and are defined by the scope of the claims, which set forth non-limiting embodiments of the disclosure.

Finally, the present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

What is claimed:

1. A method of determining exposure of a subject to a pathogen or microbe, comprising (i) incubating a liquid biological sample obtained from a subject with a bead particle comprising a core bead coupled to a plurality of antibodies bound to an antigen derived from a pathogen or microbe of interest, such that the antigen is presented on the outside of the bead particle and any antibodies in the liquid biological sample that recognize the antigen can bind to the bead particle; (ii) washing the bead particle; (iii) incubating the bead particle with a detectably labeled antibody that binds to an antibody of the species of the subject; and determining the presences or absence of the detectably labeled antibody, wherein the presence of the detectable label indicates the subject was exposed to the pathogen or microbe of interest and the absence of the detectable label indicates the subject was not exposed to the pathogen or microbe of interest.

2. The method of claim 1, wherein the subject is a human.

3. The method of claim 1 or 2, wherein the detectably labeled antibody specifically binds to IgG and/or IgM.

4. The method of any one of claims 1-3, wherein the detectable label is selected from among a fluorophore, a pigment, a radioactive isotope, a chemiluminescent molecule, a chromophore, an electron dense label, an enzyme, a dye, a metal, biotin, avidin, streptavidin, and a hapten.

5. The method of any one of claims 1-4, wherein the pathogen or microbe of interest is a virus, bacteria, or fungi.

6. The method of any one of claims 1-5, wherein the antibodies coupled to the core bead are anti-His antibodies and the antigen is His-tagged.

7. The method of any one of claims 1-6, wherein the antigen was produced recombinately from a bacterial host.

8. The method of any one of claims 1-7, wherein the core bead is a polystyrene microparticle.

9. The method of any one of claims 1-8, wherein the liquid biological sample is selected from among blood, serum, and plasma.

10. The method of any one of claims 1-8, wherein the liquid biological sample is diluted serum.

11. The method of any one of claims 1-10, wherein the amount of the detectable label determined to be present in the liquid biological sample correlates to the amount of anti-pathogen or anti-microbe IgG and/or IgM in the liquid biological sample.

12. A bead particle comprising a microparticle coupled to at least one antibody or a plurality or antibodies bound to an antigen derived from a pathogen or microbe of interest; wherein the antigen comprises a tag sequence and wherein the antibody or antibodies specifically bind to the tag sequence.

13. The bead particle of claim 12, wherein the microparticle is a polystyrene microparticle.

14. The bead particle of claim 12 or 13, wherein the pathogen or microbe of interest is a virus, bacteria, or fungi.

15. The bead particle of claim 12 or 13, wherein the tag sequence is a His-tag.

16. The bead particle of any one of claims 12-15, wherein the antigen was produced recombinately from a bacterial host.

17. A kit comprising the bead particle of any one of claims 12-16.

18. The kit of claim 17 further comprising a detectably labeled antibody that specifically binds to IgG and/or IgM, wherein the IgG and/or IgM are, optionally, human IgG and/or human IgM.

19. The kit of claim 18, wherein the detectably labeled antibody binds to human IgG and/or IgM.

20. The kit of claim 18 or 19, wherein the detectable label is selected from among a fluorophore, a pigment, a radioactive isotope, a chemiluminescent molecule, a chromophore, an electron dense label, an enzyme, a dye, a metal, biotin, avidin, streptavidin, and a hapten.

21. A method of determining exposure of a subject to Borrelia miyamotoi, comprising (i) incubating a liquid biological sample obtained from a subject with a bead particle comprising a core bead coupled to a plurality of antibodies bound to a B. miyamotoi glycerophosphodiester phosphodiesterase (glpQ), such that any anti-glpQ antibodies in the liquid biological sample can bind to the glpQ on the bead particle; (ii) washing the bead particle; (iii) incubating the bead particle with a detectably labeled antibody that binds to an antibody of the species of the subject; and determining the presences or absence of the detectably labeled antibody, wherein the presence of the detectable label indicates the subject was exposed to B. miyamotoi and the absence of the detectable label indicates the subject was not exposed to B. miyamotoi.

22. The method of claim 21, wherein the subject is a human.

23. The method of claim 21 or 22, wherein the detectably labeled antibody specifically binds to IgG and/or IgM.

24. The method of any one of claims 21-23, wherein the detectable label is selected from among a fluorophore, a pigment, a radioactive isotope, a chemiluminescent molecule, a chromophore, an electron dense label, an enzyme, a dye, a metal, biotin, avidin, streptavidin, and a hapten.

25. The method of any one of claims 21-24, wherein the antibodies bound to the B. miyamotoi glpQ specifically bind to B. miyamotoi glpQ.

26. The method of any one of claims 21-24, wherein the antibodies bound to the B. miyamotoi glpQ are anti-histidine antibodies and the B. miyamotoi glpQ is His-tagged.

27. The method of any one of claims 21-26, wherein the B. miyamotoi glpQ was produced recombinately from a bacterial host.

28. The method of any one of claims 21-27, wherein the core bead is a polystyrene microparticle.

29. The method of any one of claims 21-28, wherein the liquid biological sample is selected from among blood, serum, and plasma.

30. The method of any one of claims 21-28, wherein the liquid biological sample is diluted serum.

31. The method of any one of claims 21-30, wherein the amount of the detectable label determined to be present in the liquid biological sample correlates to the amount of anti-B. miyamotoi IgG and/or IgM in the liquid biological sample.

32. A bead particle comprising a microparticle coupled to a plurality of antibodies bound to a B. miyamotoi glycerophosphodiester phosphodiesterase (glpQ).

33. The bead particle of claim 32, wherein the microparticle is a polystyrene microparticle.

34. The bead particle of claim 32 or 33, wherein the antibodies bound to the B. miyamotoi glpQ specifically bind to B. miyamotoi glpQ.

35. The bead particle of claim 32 or 33, wherein the antibodies bound to the B. miyamotoi glpQ are anti-histidine antibodies and the B. miyamotoi glpQ is His-tagged.

36. The bead particle of any one of claims 32-35, wherein the B. miyamotoi glpQ was produced recombinately from a bacterial host.

37. A kit comprising the bead particle of any one of claims 32-36.

38. The kit of claim 37 further comprising a detectably labeled antibody that specifically binds to IgG and/or IgM, wherein the IgG and/or IgM are, optionally, human IgG and/or human IgM.

39. The kit of claim 38, wherein the detectably labeled antibody binds to human IgG and/or IgM.

40. The kit of claim 38 or 39, wherein the detectable label is selected from among a fluorophore, a pigment, a radioactive isotope, a chemiluminescent molecule, a chromophore, an electron dense label, an enzyme, a dye, a metal, biotin, avidin, streptavidin, and a hapten.

41. A method of determining exposure of a subject to hepatitis D virus (HDV), comprising (i) incubating a liquid biological sample obtained from a subject with a bead particle comprising a core bead coupled to a plurality of antibodies bound to a HDV antigen, such that any antibodies in the liquid biological sample that are capable of binding to the HDV antigen can bind to the HDV antigen on the bead particle; (ii) washing the bead particle; (iii) incubating the bead particle with a detectably labeled antibody that binds to an antibody of the species of the subject; and determining the presences or absence of the detectably labeled antibody, wherein the presence of the detectable label indicates the subject was exposed to HDV and the absence of the detectable label indicates the subject was not exposed to HDV.

42. The method of claim 41, wherein the subject is a human.

43. The method of claim 41 or 42, wherein the detectably labeled antibody specifically binds to IgG and/or IgM.

44. The method of any one of claims 41-43, wherein the detectable label is selected from among a fluorophore, a pigment, a radioactive isotope, a chemiluminescent molecule, a chromophore, an electron dense label, an enzyme, a dye, a metal, biotin, avidin, streptavidin, and a hapten.

45. The method of any one of claims 41-44, wherein the antibodies bound to the HDV antigen specifically bind to the HDV antigen.

46. The method of any one of claims 41-44, wherein the antibodies bound to the HDV antigen are anti-histidine antibodies and the HDV antigen is His-tagged.

47. The method of any one of claims 41-46, wherein the HDV antigen was produced recombinately from a bacterial host.

48. The method of any one of claims 41-47, wherein the core bead is a polystyrene microparticle.

49. The method of any one of claims 41-48, wherein the liquid biological sample is selected from among blood, serum, and plasma.

50. The method of any one of claims 41-48, wherein the liquid biological sample is diluted serum.

51. The method of any one of claims 41-50, wherein the amount of the detectable label determined to be present in the liquid biological sample correlates to the amount of anti-HDV IgG and/or IgM in the liquid biological sample.

52. A bead particle comprising a microparticle coupled to a plurality of antibodies bound to a hepatitis D virus (HDV) antigen.

53. The bead particle of claim 52, wherein the microparticle is a polystyrene microparticle.

54. The bead particle of claim 52 or 53, wherein the antibodies bound to HDV antigen specifically bind to the HDV antigen.

55. The bead particle of claim 52 or 53, wherein the antibodies bound to the HDV antigen are anti-histidine antibodies and the HDV antigen is His-tagged.

56. The bead particle of any one of claims 52-55, wherein the HDV antigen was produced recombinately from a bacterial host.

57. A kit comprising the bead particle of any one of claims 52-56.

58. The kit of claim 57 further comprising a detectably labeled antibody that specifically binds to IgG and/or IgM, wherein the IgG and/or IgM are, optionally, human IgG and/or human IgM.

59. The kit of claim 58, wherein the detectably labeled antibody binds to human IgG and/or IgM.

60. The kit of claim 58 or 59, wherein the detectable label is selected from among a fluorophore, a pigment, a radioactive isotope, a chemiluminescent molecule, a chromophore, an electron dense label, an enzyme, a dye, a metal, biotin, avidin, streptavidin, and a hapten.
